(19) United States
(12) Patent Application Publication
(10) Pub. No.: US 2003/0027755 A1
(43) Pub. Date: Feb. 6, 2003

(54) Compositions and methods for the rescue of white matter

(76) Inventors: Jian Guan, Auckland (NZ); Alistair J. Gunn, Auckland (NZ); Laura Bennet, Auckland (NZ); James J. Egan, Cambridge, MA (US)

Correspondence Address: HELLER EHRMAN WHITE & MCAULIFFE LLP, 275 MIDDLEFIELD ROAD, MENLO PARK, CA, 94025-3506, US

(21) Appl. No.: 10/013,812

(22) Filed: Dec. 7, 2001

Related U.S. Application Data
(60) Provisional application No. 60/254,349, filed on Dec. 8, 2000.
(60) Provisional application No. 60/287,668, filed on Apr. 30, 2001.

(51) Int. Cl. A61K 38/18

(52) U.S. Cl. 514/12

(57) ABSTRACT

Methods and compositions for the protection of white matter of the central nervous system. Specifically, the use of IGF-I, its analogs and mimetics, and the use of IGF-I, its analogs and mimetics in combination with interferons including beta 1 and consensus interferons, to stimulate glial cells such as mature astrocytes to promote remyelination to treat neuronal disease and injury, such as result from, for example, hypoxia, ischemia, trauma, degenerative and demyelinating diseases.

### CROSS-REFERENCE TO RELATED APPLICATIONS

This application claims the priority under 35 USC 119(e) of Provisional Application No. 60/254,349, filed Dec. 8, 2000, and Provisional Application No. 60/287,668, filed Apr. 30, 2001. These applications are incorporated herein by reference.

### FIELD OF THE INVENTION

This invention is directed to compositions and methods for the use of insulin-like growth factor-I (IGF-I), its analogs and mimetics in the treatment of neuronal injury and disease. Specifically, it is directed to the use of IGF-I, its analogs and mimetics to stimulate myelin production in mature astrocytes to treat neuronal disease and injury.

### BACKGROUND

IGF-I is a 70 amino acid polypeptide. The human form of IGF-I is a 7649-dalton polypeptide with a pI of 8.4 (Rinderknecht.et al., (1976) “Polypeptides with nonsuppressible insulin-like and cell-growth promoting activities in human serum: isolation, chemical characterization, and some biological properties of forms I and II”, Proc. Nat'l Acad. Sci. USA, 73: 2365-2369.). IGF-I is found naturally in human body fluids, for example, blood and human cerebral spinal fluid. Most tissues, and especially the liver, produce IGF-I together with specific IGF-binding proteins (IGFBPs). IGF-I production is under the dominant stimulatory influence of growth hormone (GH), and some of the IGFBPs are also increased by GH (Tanner et al., (1977) “Comparative rapidity of response of height, limb muscle and limb fat to treatment with human growth hormone in patients with and without growth hormone deficiency”, Acta Endocrinologica 84: 681-696). IGF-I has been isolated from human serum and produced recombinantly (e g. European Published Patent Applications Nos. 123,228 and 128,733).

The IGFBPs are a family of at least 6 proteins (Jones et al., (1995) “Insulin-like growth factors and their binding proteins: biological actions”, Endocrine Rev. 16: 3-34; Bach et al., (1995) “Insulin-like growth factor binding proteins”, Diabetes Rev. 3: 38-61), with other related proteins also possibly binding the IGFs. The IGFBPs bind IGF-I and IGF-II with various affinities and specificities (Jones et al., 1995). For example, IGFBP-3 binds IGF-I and IGF-II with a similar affinity, whereas IGFBP-2 and IGFBP-6 bind IGF-II with a much higher affinity than they bind IGF-I (Bach et al., 1995).

In contrast to many other growth factors, the IGFs are present in high concentrations in the circulation, but only a small fraction of the IGFs is not protein bound. For example, it is generally known that in humans and rodents less than 1% of the IGFs in blood is in a “free” or unbound form (Juul et al., (1996) “Serum concentrations of free and total insulin-like growth factor-I, IGF binding proteins −1 and −3 and IGFBP-3 protease activity in boys with normal or precocious puberty”, Clin. Endocrinology 44: 515-523 .; Hizuka et al., (1991) “Measurement of free form of insulin-like growth factor I in human plasma” Growth Regulation 1: 51-55; Hasegawa et al., (1995) “The free form of insulin-like growth factor I increases in circulation during normal human pregnancy”, J Clin. Endocrinology and Metabolism 80: 3284-3286). The overwhelming majority of the IGFs in blood circulate as part of a non-covalently associated ternary complex composed of IGF-I or IGF-II, IGFBP-3, and a large protein termed the acid-labile subunit. This complex is composed of equimolar amounts of each of the three components. The ternary complex of an IGF, IGFBP-3 and acid-labile subunit has a molecular weight of approximately 150,000 daltons, and it has been suggested that the function of this complex in the circulation may be to serve as a reservoir and buffer for IGF-I and IGF-II, preventing rapid changes in free IGF-I or IGF-II.

The IGF system is also composed of membrane-bound receptors for IGF-I, IGF-II and insulin. The Type I IGF receptor is closely related to the insulin receptor in structure and shares some of its signaling pathways (Jones et al., 1995). The IGF-II receptor is a clearance receptor that appears not to transmit an intracellular signal (Jones et al., 1995). Since IGF-I and IGF-II bind to the Type 1 IGF-I receptor with a much higher affinity than to the insulin receptor, it is most likely that most of the effects of IGF-I and IGF-II are mediated by the Type I IGF receptor (Ballard et al., (1994) “Does IGF-I ever act through the insulin receptors”, The Insulin-like Growth Factors and Their Regulatory Proteins. Baxter, Gluckman, and Rosefeld, eds., Amsterdam: Elsevier, pp. 131-138.

Various biological activities of IGF-I have been identified. For example, IGF-I is reported to lower blood glucose levels in humans (Guler et al., (1987) “Short term metabolic effects of recombinant human insulin-like growth factor I in healthy adults”, New Eng. J Med. 317: 137-140). Additionally, IGF-I promotes growth in several metabolic conditions characterized by low IGF-I levels, such as hypophysectomized rats (Skottner et al., (1987) “Recombinant human insulin-like growth factor: testing the somatomedin hypothesis in hypophysectomized rats”, J Endocrinology 112: 123-132), diabetic rats (Scheiwiller et al., (1986) “Growth restoration of insulin deficient diabetic rats by recombinant human insulin like growth factor I”, Arch. Dis. Child. 65: 1017-1020), and dwarf rats (Skottner et al., (1989) “Growth responses in a mutant dwarf rat to human growth hormone and recombinant human insulin-like growth factor I”, Endocrinology 124: 2519-2526). The kidney weight of hypophysectomized rats increases substantially upon prolonged infusions of IGF-I subcutaneously (Guler et al., (1989) “Effects of recombinant insulin like growth factor I on insulin secretion and renal function in normal human subjects”, Proc. Nat'l Acad. Sci. USA 86: 2868-2872). The anabolic effect of IGF-I in rapidly growing neonatal rats was demonstrated in vivo (Philipps et al., (1988) “The effects of biosynthetic insulin like growth factor I. Supplementation on somatic growth, maturation, and erythropoiesis on the neonatal rat”, Ped. Res. 23: 298-305). In underfed, stressed, ill, or diseased animals, IGF-I levels are well known to be depressed.

IGF-I is thought to play a paracrine role in the developing and mature brain (Werther et al., (1990) “Localization of insulin like growth factor I mRNA in rat brain by in situ hybridisation-relationship of IGF-I receptors”, Mol Endocrinology 4: 773-778). In vitro studies indicate that IGF-I is a potent non-selective trophic agent for several types of neurons in the central nervous system (CNS) (Knusel et al., (1990) “Selective and nonselective stimulation of central cholinergic and dopaminergic development in vitro by nerve growth factor, basic fibroblast growth factor, epidermal growth factor, insulin and the insulin-like growth factors I and II”, J Neurosci. 10: 558-570; Svrzic et al., (1990) “Insulin-like growth factor 1 supports embryonic nerve cell survival”, Biochem. Biophys. Res. Commun. 172: 54-60), including dopaminergic neurons (Knusel et al., 1990), and for oligodendrocytes (McMorris et al., (1988) “Insulin-like growth factor I promotes cell proliferation and oligodendroglial commitment in rat glial progenitor cells developing in vitro”, J. Neurosci. Res. 21: 199-209; McMorris et al., (1986) “Insulin like growth factor I/somatomedin C: a potent inducer of oligodendrocyte development”, Proc. Nat'l Acad Sci. USA 83(3):822-826; Mozell et al., (1991) “Insulin-like growth factor I stimulates oligodendrocyte development and myelination in rat brain aggregate cultures”. J Neurosci. Res. 30: 382-390). Methods for enhancing the survival of cholinergic neuronal cells by administration of IGF-I have been described in U.S. Pat. Nos. 5,093,317 and 5,652,214.

Both the central nervous system (CNS) and peripheral nervous system (PNS) contain both neuronal cells and glial cells. Although neurons are thought to produce and carry nervous impulses, and glial cells are thought to act in a more passive, supporting role, glial cells are important to the survival and function of the nervous system. There are several types of glia, including oligodendrocytes, Schwann cells, astrocytes, satellite cells, microglia, and others. Oligodendrocytes in the CNS and Schwann cells in the PNS form myelin sheaths around the axons of neurons, which greatly enhances neuronal communication. Astrocytes in the CNS and satellite cells in the PNS provide nourishment and structural support to neurons, remove metabolic waste products, and are critical in the establishment and functioning of the blood-brain barrier. Oligodendrocytes and astrocytes in the CNS and Schwann cells and satellite cells in the PNS are important in neuronal injury and disease. Microglia share some of the functions of astrocytes and satellite cells, and are also important in response to injury and disease.

IGF-I receptors are wide spread in the CNS (Bohannon et al., (1988) “Localization of binding sites for insulin-like growth factor-1 (IGF-1) in the rat brain by quantitative autoradiography”, Brain Res. 444: 205-213; Bondy et al., (1992) “Cellular pattern of type-I insulin like growth factor gene expression during maturation of the rat brain: comparison with insulin-like growth factors I and II”, Neuroscience 46: 909-923) occurring on both glia (Kiess et al., (1989) “Rat C6 glial cells synthesise insulin like growth factor I (IGF-I) and express IGF-I receptors and IGF-II/mannose 6-phosphate receptors”, Endocrinology 124: 1727-1736) and neurons (Sturm et al., (1989) “Insulin like growth factor receptors and binding protein in rat neuroblastoma cells”, Endocrinology 124: 388-396). These receptors mediate the anabolic and somatogenic effects of IGF-I and have a higher affinity for IGF-I compared to insulin (Hill et al., (1986) “Autoradiographic localization of insulin receptors in rat brain: prominence in olfactory limbic areas”, Neuroscience 17: 1127-1138; Lesniak et al., (1988) “Receptors for insulin-like growth factor s I and II: autoradiographic localization in rat brain and comparison to receptors for insulin”, Endocrinology 123: 2089-2099). From 3 days after injury, greatly increased levels of IGF-I are produced particularly in the developing CNS (Gluckman et al., (1992) “A role for IGF-1 in the rescue of CNS neurons following hypoxic-ischemic injury”, Biochem. Biophys. Res. Commun. 182: 593-599; Yamaguchi et al., (1991) “Increase of extracellular insulin-like growth factor I (IGFI) concentration following electrolytical lesion in rat hippocampus”, Neurosci. Lett. 128: 373-376). The effect of IGF-I as a central neuroprotectant when administered after an insult (Gluckman et al., 1992) suggests a mode of action involving interference with the activated processes leading to cell death. Endogenous and exogenous IGF-I stimulate peripheral nerve regeneration (Karj e et al (1989) “Insulin like growth factor I (IGF-1) stimulates regeneration of the rat sciatic nerve”, Brain Res. 486: 396-398). IGF-I has been shown to enhance ornithine decarboxylase activity in normal rat brains (U.S. Pat. No. 5,093,317).

Interferons (IFNs) are a subclass of cytokines that collectively have anti-viral, anti-microbial and anti-proliferative functions and also have roles in cytokine regulated immune activities (reviewed in Weinstock-Guttman et al., (1995) “The interferons: biological effects, mechanisms of action, and uses in multiple sclerosis”, Ann. Neurology 37: 7-15). Many cell types in the body produce interferons and high affinity receptors are found on most cells. There are two main types of interferons; type I consisting of alpha, beta and omega classes and type II made up of the gamma class.

Type I interferons consist of more that 16 subclasses of alpha interferons and beta and omega interferon. Type I's bind to a cell surface receptor and set in motion a complex series of events that lead to the induction of anti-proliferative and anti-viral activity, immunomodulatory actions, cytokine induction and the regulation of HLA classes I and II (Pestka et al., (1987) “Interferons and their actions”, Ann. Rev. Biochem. 56: 727-777). All the alpha interferons have biological effects that are similar, but not all these effects are shared by each subtype and extent of activity varies. Beta interferon (IFN-β1b, Betaseron™/Betaferon™; IFN-β1a, Avonex™) is used as a treatment for multiple sclerosis (reviewed in Compston, (1998) “Treatment and management of multiple sclerosis” in McAlpine 's Multiple Sclerosis, Compston, Ebers, Lassmann, McDonald, Matthews, and H Wekerle, eds. London: Churchill Livingstone. 469-472; 474-486). Gamma interferon also has anti-viral activity but this is weaker than type-I interferons. It can also be distinguished from type I's by different immune functions, for example macrophage activation.

Consensus interferon (for example Infergen™, Amgen) is a non-naturally occurring type-I interferon that was bioengineered from a consensus sequence of interferons and developed for the treatment of chronic hepatitis C (U.S. Pat. Nos. 6,172,046 and 6,207,145). Human interferon polypeptides with amino acid sequences that have commonly or mostly amino acids located at each position among endogenous alpha interferon subtype polypeptides called consensus interferons are disclosed in U.S. Pat. No. 4,695,623, U.S. Pat. No. 4,897,471 and U.S. Pat. No. 5,541,293. The sequences disclosed are designated IFN-con₁, IFN-con₂, and IFN-con₃. Consensus interferon has been demonstrated to have greater biological activity in many instances than naturally occurring interferons.

Injury of immature white matter is well known to be the dominant cause of neural handicap in very premature infants (Inder et al., (1999) “Periventricular white matter injury in the premature infant is followed by reduced cerebral cortical gray matter volume at term”, Ann. Neurol. 46: 755-760). In contrast, the causes and treatment of white matter damage in the more mature infant, and the adult, have been relatively neglected (Petty et al., (2000) “White matter ischaemia”, Brain Res. Rev. 31: 58-64). This in part is due to a general scientific consensus that white matter was less vulnerable to injury than grey matter (Marcoux et al., (1982) “Differential regional vulnerability in transient focal cerebral ischemia”, Stroke 13: 339-346). However, recent imaging data show that cerebral white matter injury also contributes to developmental disability after perinatal hypoxic-ischemic injury at term (Mercuri et al., (1999) “Neonatal neurological examination in infants with hypoxic ischaemic encephalopathy: correlation with MRI findings”, Neuropediatrics 30: 83-89; Okumura et al., (1997) “MRI findings in patients with spastic cerebral palsy. I: Correlation with gestational age at birth”, Dev. Med. Child Neurol. 39: 363-368).

Experimentally, it is now increasingly recognized that differentiated oligodendrocytes and myelinated axons are also vulnerable to ischemic injury (Jelinski et al., (1999) “Preferential injury of oligodendroblasts by a short hypoxic-ischemic insult”, Brain Res. 815: 150-153; Nedelcu et al., (1999) “Biphasic edema after hypoxic-ischemic brain injury in neonatal rats reflects early neuronal and late glial damage”, Ped. Res. 46: 297-304; Ikeda et al., (1998) “Physiologic and histologic changes in near-term fetal lambs exposed to asphyxia by partial umbilical cord occlusion”, Am. J Obstet. Gynecol. 178: 24-32; Petito et al., (1998) “Selective glial vulnerability following transient global ischemia in rat brain”, J. NeuropathoL Exp. Neurol. 57: 231-238; Mandai et al., (1997) “Ischemic damage and subsequent proliferation of oligodendrocytes in focal cerebral ischemia”, Neuroscience 77: 849-861; Pantoni et al., (1996) “Cerebral white matter is highly vulnerable to ischemia”, Stroke 27: 1641-1646). As an example, in the 7 day old rat data from magnetic resonance imaging indicates that hypoxia-ischemia led to extensive secondary glial swelling and death, which followed an earlier phase of delayed neuronal death (Nedelcu et al., 1999). In contrast, after focal ischemia in the adult rat oligodendrocyte loss developed earlier than neuronal injury (Pantoni et al., 1996). Similarly, the mildest lesion seen after asphyxia in the near-term fetal sheep was vacuolation and loss of myelin in white matter, rather than neuronal death (Ikeda et al., 1998).

The pathogenesis of demyelination after injury may be a consequence of the loss of mature oligodendrocytes (Mandai et al., 1997; Shuman et al., (1997) “Apoptosis of microglia and oligodendrocytes after a spinal cord contusion in rats”, J. Neurosci. Res. 50: 798-808), or secondary to other processes such as microglial activation or the loss of trophic support after axonal degeneration (Shuman et al., 1997). Evidence suggests that insulin-like growth factor-I (IGF-I) may reduce both primary and secondary post-ischemic white matter injury. IGF-I promotes the proliferation and differentiation of olgiodendroglia and upregulates myelin production in vitro (Ye et al., (1999) “Insulin-like growth factor 1 protects oligodendrocytes from tumor necrosis factor-alpha-induced injury”, Endocrinology 140: 3063-3072; Wilczak et al., (1997) “Insulin-like growth factor-1 receptors in normal appearing white matter and chronic plaques in multiple sclerosis”, Brain Res. 772:243-246; Dercole et al., (1996) “The role of the insulin-like growth factors in the central nervous system”, Mol. Nuerobiol. 13: 227-255; McMorris et al., (1996) “Regulation of oligodendrocyte development and CNS myelination by growth factors-prospects for therapy of demyelinating disease”, Brain Pathol. 6: 313-329; Shinar et al., (1995) “Developing oligodendroglia express mRNA for insulin-like growth factor-I, a regulator of oligodendrocyte development”, J. Neurosci. Res. 42: 516-527). It has broad, receptor-mediated anti-apoptotic effects in vitro and in vivo (Parrizas et al., (1997) “Insulin-like growth factor 1 inhibits apoptosis using the phosphatidylinositol 3′-kinase and mitogen-activated protein kinase pathways”, J. Biol. Chem. 272: 154-161; Galli et al., (1995) “Apoptosis in cerebellar granule cells is blocked by high KCl, forskolin, and IGF-1 through distinct mechanisms of action: The involvement of intracellular calcium and RNA synthesis”, J. Neurosci. 15: 1172-1179; Yin et al., (1994) “Cell death of spinal motoneurons in the chick embryo following deafferentation: rescue effects of tissue extracts, soluble proteins, and neurotrophic agents”, J. Neurosci. 14: 7629-7640), and specifically inhibits the apoptotic loss of oligodendrocytes associated with cytokine toxicity and metabolic insults (Mason et al., (2000) “Insulin-Like Growth Factor-1 inhibits mature oligodendrocyte apoptosis during primary demyelination”, J. Neurosci. 20: 5703-5708).

Experimental myelination is associated with distinctive patterns of induction of IGF-I in astrocytes and of the IGF-1 receptor in oligodendrocytes during regeneration (Hinks et al., (1999) “Distinctive patterns of PDGF-A, FGF-2, IGF-1 and TGF-beta 1 gene expression during remyelination of experimentally-induced spinal cord demyelination”, Mol. Cell. Neurosci. 14: 153-168; Komoly et al., (1992) “Insulin-like growth factor I gene expression is induced in astrocytes during experimental demyelination”, Pro.c Nat'l Acad. Sci. USA 89:1894-1898), suggesting that endogenous IGF-I may play an important role in remyelination. Consistent with this hypothesis, IGF-I is also intensely induced in reactive glia 3 to 5 days after hypoxic-ischemic injury, although the relationship with remyelination has not been examined (Lee et al., (1993) “Insulin-like growth factors and cerebral ischemia”, Ann. NY Acad. Sci. 679: 418-422; Gluckman et al., 1992). At present, little is known about the role of IGF-I in oligodendrocyte survival or cerebral demyelination after hypoxic-ischemic injury in the developing brain.

To date however, there has been no teaching that IGF-I or its analogs or mimetics have any direct effect on stimulating mature astrocytes to promote the production of myelin, nor are there are treatments currently available to prevent the loss of oligodendrocytes and cerebral demyelination that occurs in the developing brain as a consequence of hypoxic-ischemic injury.

The documents cited in this section and elsewhere in this application are incorporated herein by reference.

### SUMMARY OF THE INVENTION

Recognising the significance of these problems, it is an object of the present invention to provide new approaches to therapy for brain injury and disease, and to provide compositions and methods effective to treat brain injury and disease. In particular, it is an object of the present invention to provide compositions and methods for treating brain injury and disease comprising administering IGF-I, IGF-I analogs, and IGF-I mimetics (IGF-I compounds) effective to restore myelination of axons in animals. For example, administration of IGF-I compounds is effective to stimulate myelin production in oligodendrocytes and to stimulate the promotion of remyelination by mature astrocytes after hypoxic-ischemic injury to the brain or as a therapy for multiple sclerosis.

The invention relates to a method of restoring myelination of axons in an animal in need of restored myelination due to neural injury or disease, comprising administering a therapeutic amount of an IGF-I compound, where an IGF-I compound comprises IGF-I, a biologically active IGF-I analog, a biologically active IGF-I mimetic, a compound that increases the concentration of IGF-I, or a compound that increases the concentration of IGF-I analogs, effective to restore myelination of axons in an animal. In one aspect of the invention, the method of restoring myelination of axons comprising administering a therapeutic amount of an IGF-I compound to stimulate astrocytes to promote remyelination. In another aspect of the invention, the method of restoring myelination of axons comprising administering a therapeutic amount of an IGF-I compound to stimulate oligodendrocytes to produce myelin.

In other aspects of the method of restoring myelination of axons to an animal in need of restored myelination due to neural injury or disease, the neural injury or disease comprises a disorder selected from the group consisting of trauma, toxin exposure, asphyxia or hypoxia-ischemia, perinatal hypoxic-ischemic injury, injury to or disease of the white matter of the central nervous system, acute brain injury, chronic neurodegenerative disease, and demyelinating diseases and disorders. In a preferred embodiment of the invention, the chronic neurodegenerative disease is multiple sclerosis. In another preferred embodiment of the invention, the demyelinating diseases and disorders are selected from the group consisting of inflammatory involvement: acute disseminated encephalomyelitis, optic neuritis, transverse myelitis, Devic's disease, the leucodystrophies; non-inflammatory involvement: progressive multifocal leukoencephalopathy, and central pontine myelinolysis.

In another aspect of the invention, the method of restoring myelination of axons in an animal in need of restored myelination further comprises administering a therapeutic amount of an IGF-I compound in combination with an interferon. In one aspect of the invention, the method of restoring myelination of axons comprising administering a therapeutic amount of an IGF-I compound in combination with an interferon to stimulate astrocytes to promote remyelination. In another aspect of the invention, the method of restoring myelination of axons comprising administering a therapeutic amount of an IGF-I compound in combination with an interferon to stimulate oligodendrocytes to produce myelin. The interferon may be any interferon, and may be an interferon selected from the group consisting of interferon-alpha, interferon-beta, interferon-omega, consensus-interferon and combinations thereof. In preferred embodiments, the interferon comprises an interferon beta. In a most preferred embodiment, the interferon comprises interferon β1b (Betaseron™). In a further most preferred embodiment, the interferon comprises consensus interferon (Infergen™, interferon alfacon-1).

In another aspect of the method of restoring myelination of axons in an animal in need of restored myelination, the step of administering a therapeutic amount of an IGF-I compound further comprises introducing a nucleic acid encoding an IGF-I compound into the animal.

In an aspect of the invention, the method of restoring myelination of axons in an animal in need of restored myelination further comprises the administration of a therapeutically effective amount of a growth-promoting agent, where a growth-promoting agent comprises growth hormone, growth hormone analogs, growth hormone mimetics, agents that increase the concentration of growth hormone in the blood of an animal, and growth hormone secretagogues.

In another aspect of the invention, a kit is provided, where the kit comprises an IGF-I compound formulated in a pharmaceutically acceptable buffer, a container for holding said IGF-I compound formulated in a pharmaceutically acceptable buffer, and instructions. In a further aspect of the invention, the kit may further comprise a compound selected from the group consisting of growth hormone, a growth hormone releasing protein, a growth hormone releasing hormone, a growth hormone secretagogue, and a growth hormone complexed with a growth hormone binding protein. In yet a further aspect of the invention, the kit may further comprise a compound selected from the group consisting of an IGF binding protein, an IGF-I compound complexed to an IGF binding protein, insulin, and a hypoglycemic agent.

In yet another aspect of the invention, the method of restoring myelination of axons in an animal in need of restored myelination due to neural injury or disease, comprises administration of a therapeutic amount of an IGF-I compound in the period from the time of the central nervous system injury to about 100 hours after the injury.

In yet another aspect of the invention, the method of restoring myelination of axons in an animal in need of restored myelination due to neural injury or disease, comprises administration of a therapeutic amount of an IGF-I compound in combination with an interferon in the period from the time of the central nervous system injury to about 100 hours after the injury. In a preferred embodiment, the interferon is an interferon beta. In a most preferred embodiment, the interferon is interferon β1b (Betaseron™). In a further most preferred embodiment, the interferon comprises consensus interferon (Infergen™, interferon alfacon-1).

In another aspect of the invention, the method of restoring myelination of axons in an animal in need of restored myelination due to neural injury or disease, comprises administration of a therapeutic amount of an IGF-I compound at least once in the period from the time of the central nervous system injury to about 8 hours subsequently.

In still another aspect of the invention, the method of restoring myelination of axons in an animal in need of restored myelination due to neural injury or disease, comprises administration of a therapeutic amount of an IGF-I compound in combination with an interferon at least once in the period from the time of the central nervous system injury to about 8 hours subsequently. In a preferred embodiment, the interferon is an interferon beta. In a most preferred embodiment, the interferon is interferon β1b (Betaseron™). In a further most preferred embodiment, the interferon comprises consensus interferon (Infergen™, interferon alfacon-1).

In a further aspect of the invention, the method of restoring myelination of axons in an animal in need of restored myelination due to neural injury or disease, comprises administration of a therapeutic amount of an IGF-I compound in an amount from about 0.1 to about 1000 μg of IGF-I per 100 g of body weight of the animal.

In still a further aspect of the invention, the method of restoring myelination of axons in an animal in need of restored myelination due to neural injury or disease, comprises administration of a therapeutic amount of an IGF-I compound in combination with an interferon in an amount from about 0.1 to about 1000 μg of IGF-I per 100 g of body weight of the animal. In a preferred embodiment, the interferon is an interferon beta. In a most preferred embodiment, the interferon is interferon β1b (Betaseron™). In a further most preferred embodiment, the interferon comprises consensus interferon (Infergen™, interferon alfacon-1).

In a preferred embodiment of the method of restoring myelination of axons in an animal in need of restored myelination due to neural injury or disease, comprising administration of an IGF-I compound, the IGF-I compound is a biologically active analog of IGF-I selected from the group consisting of insulin-like growth factor 2 (IGF-2) and truncated IGF-I (des 1-3 IGF-I).

In another preferred embodiment of the method of restoring myelination of axons in an animal in need of restored myelination due to neural injury or disease, comprising administration of an IGF-I compound, the IGF-I compound is a biologically active mimetic of IGF-I. In a more preferred embodiment, the biologically active mimetic of IGF-I is selected from the group consisting of IGFBP-1 binding peptide p1-01 and insulin-like growth factor agonist molecules.

In a further preferred embodiment of the method of restoring myelination of axons in an animal in need of restored myelination due to neural injury or disease, comprising administration of an IGF-I compound, the IGF-I compound is administered to the animal through a shunt into a ventricle of the animal.

In a further preferred embodiment of the method of restoring myelination of axons in an animal in need of restored myelination due to neural injury or disease, comprising administration of an IGF-I compound, the IGF-I compound is administered to the animal by peripheral administration.

In a first aspect, the invention provides a method of treatment for stimulating mature astrocytes to promote myelin production after hypoxic-ischemic injury including the step of increasing the active concentration of the IGF-I and/or the concentration of analogues of IGF-I in the CNS of mammals.

In a further aspect, the invention provides for a method of treatment for myelin loss incurred as a result of neurological damage caused by multiple sclerosis, said method comprising the step of increasing the effective amount of IGF-I or analog thereof or mimetic thereof within the CNS of said patient.

Most preferably, it is the effective amount of IGF-I itself which is increased within the CNS of the mammal. This can be effected by direct administration of IGF-I and indeed this is preferred. However, the administration of compounds which indirectly increase the effective amount of IGF-I (for example a pro-drug which, within the patient is cleaved to release IGF-I) is in no way excluded.

The active compound (IGF-I or its analog or its mimetic) can be administered alone, or as is preferred, as part of a pharmaceutical composition.

The composition can be administered directly to the CNS. The latter route of administration can involve, for example, lateral cerebro-ventricular injection, focal injection, or a surgically inserted shunt into the lateral cerebro-ventricle of the brain of the patient.

Conveniently, the stimulation and promotion of myelin production in oligodendrocytes and the support, stimulation and promotion of remyelination by mature astrocytes is promoted through the administration of IGF-I compounds in the prophylaxis or therapy of neurodegenerative diseases such as multiple sclerosis.

### DESCRIPTION OF THE INVENTION

As indicated above, the present invention is broadly based upon the applicants' surprising finding that IGF-I compounds are capable of promoting myelin production after hypoxic-ischemic injury and as a consequence of multiple sclerosis. This stimulation of myelin production is achieved through increasing the effective concentration or amount of IGF-I or an IGF-I analog or an IGF-1 mimetic in the CNS of a patient.

As used herein, an IGF-I compound is a compound with biological activity similar or identical to the biological activity of IGF-I; IGF-I compounds comprise IGF-I, biologically active IGF-I analogs, biologically active IGF-I mimetics, and compounds that increase the concentration of IGF-I and IGF-I analogs in an animal. IGF-I compounds include insulin-like growth factor agonist molecules such as peptide fragments and truncated portions of longer IGF-I compounds as well as other chemical and biological analogs and mimetics. Examples of IGF-I compounds may be found, for example, in U.S. Pat. Nos. 5,420,112; 5,652,214; and 6,121,416.

By “IGF-I analog” is meant any naturally occurring analogues of IGF-I or variants thereof which are capable of effectively binding to the IGF-I receptors in the CNS and of stimulating an equivalent myclin producing effect in mature astrocytes.

By “IGF-I mimetic” is meant any compound that prevents the interaction of IGF with any of its binding proteins and does not prevent interaction of IGF-I with a human IGF receptor. These IGF mimetic compounds include peptides, and increase serum and tissue levels of active IGFs in a mammal. For example, see U.S. Pat. No. 6,121,416; and Lowman et al., (1998) “Molecular mimics of insulin-like growth factor 1(IGF-1) for inhibiting IGF-1: IGF-binding protein interactions”, Biochemistry 37: 8870-8878, and references therein.

By “insulin-like growth factor agonist molecules” is meant a molecule affective to activate insulin-like growth factor receptors, and includes peptide fragments and truncated portions of longer IGF-I compounds as well as other chemical and biological analogs and mimetics. Examples of insulin-like growth factor agonist molecules may be found, for example, in U.S. Pat. No. 6,121,416.

As used herein, “interferon” comprises the naturally-occurring interferons and artificially created or produced interferons, including truncated interferons, interferon fragments, and interferon analogs and mimetics, both glycosylated and non-glycosylated, for example, consensus interferon (Interfergen™, interferon alfacon-1). By “naturally-occurring interferon” is meant any of a family of glycoproteins secreted by virus-infected cells known as interferons, which can protect non-infected cells from replication of the virus.

The compositions and methods of the invention find use in the treatment of animals, such as human patients, suffering from neural injury or disease. Still more generally, the compositions and methods of the invention find use in the induction of myelin production following insult in the form of trauma, toxin exposure, asphyxia or hypoxia-ischemia. In particular, the compositions and methods of the invention find use in the treatment of animals, such as human patients, suffering from white matter insult as the result of acute brain injury, such as perinatal hypoxic-ischemic injury; or from chronic neural injury or neurodegenerative disease, such as multiple sclerosis (MS); or from other demyelinating diseases and disorders including inflammatory involvement, such as acute disseminated encephalomyelitis, optic neuritis, transverse myelitis, Devic's disease, the leucodystrophies; non-inflammatory involvement: progressive multifocal leukoencephalopathy, central pontine myelinolysis. Patients suffering from such diseases or injuries will benefit greatly by a treatment protocol able to initiate remyelination.

Still more generally, the invention has application in the induction of myelin production following insult in the form of trauma, toxin exposure, asphyxia or hypoxia-ischemia.

It is presently preferred by the applicants that IGF-I itself be used to promote myelin production in mature astrocytes. Most conveniently, this is effected through the direct administration of IGF-I to the patient.

However, while this is presently preferred, there is no intention on the part of the applicants to exclude administration of other forms of IGF-I. By way of example, the effective amount of IGF-I in the CNS can be increased by administration of a prodrug form of IGF-I which comprises IGF-I and a carrier, IGF-I and the carrier being joined by a linkage which is susceptible to cleavage or digestion within the patient. Any suitable linkage can be employed which will be cleaved or digested to release IGF-I following administration.

Another option is for IGF-I levels to be increased through an implant which is or includes a cell line which is capable of expressing IGF-I in an active form within the CNS of the patient.

IGF-I can be administered as part of a medicament or pharmaceutical preparation. This can involve combining IGF-I with any pharmaceutically appropriate carrier, adjuvant or excipient. The selection of the carrier, adjuvant or excipient will of course usually be dependent upon the route of administration to be employed.

The administration route can vary widely. An advantage of IGF-I is that it can be administered peripherally. This means that it need not be administered directly to the CNS of the patient in order to have effect in the CNS.

Any peripheral route known in the art can be employed. These can include parenteral routes for example injection into the peripheral circulation, subcutaneous, intraorbital, ophthalmic, intraspinal, intracistemal, topical, infusion (using e.g. slow release devices or minipumps such as osmotic pumps or skin patches), implant, aerosol, inhalation, scarification, intraperitoneal, intracapsular, intramuscular, intranasal, oral, buccal, pulmonary, rectal or vaginal. The compositions can be formulated for parenteral administration to humans or other mammals in therapeutically effective amounts (eg. amounts which eliminate or reduce the patient's pathological condition) to provide therapy for the neurological diseases described above.

Two of the most convenient administration routes will be by subcutaneous injection (e.g. dissolved in 0.9% sodium chloride) or orally (in a capsule).

It will also be appreciated that it may, on occasion, be desirable to directly administer IGF-I compounds to the CNS of the patient. Again, this can be achieved by any appropriate direct administration route. Examples include administration by lateral cerebroventricular injection or through a surgically inserted shunt into the lateral cerebroventricle of the brain of the patient.

The calculation of the effective amount of IGF-I compounds to be administered is within the skill of one of ordinary skill in the art, and will be routine to those persons skilled in the art. Needless to say, the final amount to be administered will be dependent upon the route of administration and upon the nature of the neurological disorder or condition which is to be treated. A suitable dose range may for example be between about 0.04 mg to about 1000 mg of IGF-I compound per 100 g of body weight where the dose is administered centrally.

For inclusion in a medicament, IGF-I compounds can be obtained from a suitable commercial source. Alternatively, IGF-I, IGF-I analogs and IGF-I mimetics can be directly synthesized by conventional methods such as the stepwise solid phase synthesis method of Merrifield et al., 1963. Alternatively synthesis can involve the use of commercially available peptide synthesizers such as the Applied Biosystems model 430A.

If a small molecule antagonist is used as an IGF agonist, it may have cyclical effects and require, for efficacy, an administration regimen appropriate thereto, the variable concentration of IGFBP-1 in blood being an example (Jones et al., 1995). For a peptide, a preferred administration is a chronic administration of about two times per day for 4-8 weeks to reproduce the effects of IGF-I. Although injection is preferred, chronic infusion may also be employed using an infusion device for continuous, subcutaneous infusions. A small peptide may be administered orally. An intravenous bag solution may also be employed.

As a general proposition, the total pharmaceutically effective amount of the IGF agonist compound administered parenterally per dose will be in a range that can be measured by a dose response curve. For example, IGFs bound to IGFBPs or in the blood can be measured in body fluids of the mammal to be treated to determine dosing. Alternatively, one can administer increasing amounts of the IGF agonist compound to the patient and check the serum levels of the patient for IGF-I and IGF-II. The amount of IGF agonist to be employed can be calculated on a molar basis based on these serum levels of IGF-I and IGF-II.

Specifically, one method for determining appropriate dosing of the compound entails measuring IGF levels in a biological fluid such as a body or blood fluid. Measuring such levels can be done by any means, including RIA and ELISA. After measuring IGF levels, the fluid is contacted with the compound using single or multiple doses. After this contacting step, the IGF levels are re-measured in the fluid. If the fluid IGF levels have fallen by an amount sufficient to produce the desired efficacy for which the molecule is to be administered, then the dose of the molecule can be adjusted to produce maximal efficacy. This method can be carried out in vitro or in vivo. Preferably, this method is carried out in vivo, i.e. after the fluid is extracted from a mammal and the IGF levels measured, the compound herein is administered to the mammal using single or multiple doses (that is, the contacting step is achieved by administration to a mammal) and then the IGF levels are remeasured from fluid extracted from the mammal.

Another method for determining the amount of a particular IGFBP or the amount of the compound bound to a particular IGFBP in a biological fluid so that dosing of the compound can be adjusted appropriately involves:

1. contacting the fluid with 1) a first antibody attached to a solid-phase carrier, wherein the first antibody is specific for epitopes on the IGFBP such that in the presence of antibody the IGF binding sites remain available on the IGFBP for binding to the compound, thereby forming a complex between the first antibody and the IGFBP; and 2) the compound for a period of time sufficient to saturate all available IGF binding sites on the IGFBP, thereby forming a saturated complex;

2. contacting the saturated complex with a detectably labeled second antibody which is specific for epitopes on the compound which are available for binding when the compound is bound to the IGFBP; and

3. quantitatively analyzing the amount of the labeled second antibody bound as a measure of the IGFBP in the biological fluid, and therefore as a measure of the amount of the compound bound. This technique can be expanded to include a diagnostic use whereby the compound is administered to a mammal to displace an IGF from a specific IGFBP for which the compound has affinity, such as IGFBP-1 or IGFBP-3, and measuring the amount that is displaced.

The quantitative technique mentioned above using antibodies, called the ligand-mediated immunofunctional method (LIFA), is described for determining the amount of IGFBP by contact with IGF in U.S. Pat. No. 5,593,844, and for determining the amount of GHBP by contact with GH in U.S. Pat. No. 5,210,017.

Another method for determining dosing is to use antibodies to the IGF agonist or another detection method for the IGF agonist in the LIFA format. This would allow detection of endogenous or exogenous IGFs bound to IGFBP and the amount of IGF agonist bound to the IGFBP.

Another method for determining dosing would be to measure the level of “free” or active IGF in blood. For some uses the level of “free” IGF would be a suitable marker of efficacy and effective doses or dosing. For example, one method is described for detecting endogenous or exogenous IGF bound to an IGF binding protein or the amount of a compound that binds to an IGF binding protein and does not bind to a human IGF receptor bound to an IGF binding protein or detecting the level of unbound IGF in a biological fluid. This method comprises:

1. contacting the fluid with 1) a means for detecting the compound that is specific for the compound (such as a first antibody specific for epitopes on the compound) attached to a solid-phase carrier, such that in the presence of the compound the IGF binding sites remain available on the compound for binding to the IGF binding protein, thereby forming a complex between the means and the IGF binding protein; and 2) the compound for a period of time sufficient to saturate all available IGF binding sites on the IGF binding protein, thereby forming a saturated complex;

2. contacting the saturated complex with a detectably labeled second means which is specific for one or more sites on the IGF binding protein (such as a second antibody specific for epitopes on the IGFBP) which are available for binding when the compound is bound to the IGF binding protein; and

3. quantitatively analyzing the amount of the labeled means bound as a measure of the IGFBP in the biological fluid, and therefore as a measure of the amount of bound compound and IGF binding protein, bound IGF and IGF binding protein, or active IGF present in the fluid.

Given the above methods for determining dosages, in general, the amount of IGF agonist compound that may be employed can be estimated by methods well known in the art, as illustrated (e.g. by the methods shown in Example 11 and FIGS. 43 and 44 of U.S. Pat. No. 6,121,416 for IGF-I). An orally active small IGF agonist would have a molecular weight of approximately 500 daltons, compared to 7500 daltons for IGF-I and IGF-II. Assuming the IGF agonist is 16-fold less able to bind to IGFBPs than IGF-I or IGF-II, then equal weights of IGF-I or IGF-II and these molecules could be equally effective, so that doses from about 10 μg/kg/day to about 200 μg/kg/day might be used, based on patient body weight, although, as noted above, this will be subject to a great deal of therapeutic discretion.

A further method is provided to estimate the distribution of IGFs on specific IGFBPs (e.g. on IGFBP-1 or IGFBP-3 using the LIFA format).

The compound is suitably administered by a sustained-release system. Suitable examples of sustained-release compositions include semi-permeable polymer matrices in the form of shaped articles (e.g. films, or microcapsules). Sustained-release matrices include polylactides (U.S. Pat. No. 3,773,919; European Published Application No. 58,481), copolymers of L-glutamic acid and gamma-ethyl-L-glutamate (Sidman et al., (1983) “Controlled release of macromolecules and pharmaceuticals from synthetic polypeptides based on glutamic acid”, Biopolymers B: 547-556), poly(2-hydroxyethyl methacrylate) (Langer et al., (1981) “Biocompatibility of polymeric delivery systems for macromolecules”, J. Biomed. Mater. Res. 15: 267-277), ethylene vinyl acetate (Langer et al., 1981), or poly-D-(−)-3-hydroxybutyric acid (European Published Patent Application No. 133,988). Sustained-release compositions also include a liposomally entrapped compound. Liposomes containing the compound are prepared by methods known per se, for example, in U.S. Pat. No. 3,218,121; Hwang et al., (1980) “Hepatic uptake and degradation of unilamellar sphingomyelin/cholesterol liposomes: a kinetic study”, Proc. Nat'l Acad. Sci. USA 77: 4030-4034; European Published Patent Application Nos. 52,322; 36,676; 88,046; 143,949; and 142,641; Japanese Published Patent. Application No. 118008/1983; U.S. Pat. Nos. 4,485,045 and 4,544,545; and European Published Patent Application No. 102,324. Ordinarily, the liposomes are of the small (from or about 20 to 80 nm) unilamellar type in which the lipid content is greater than about 30 mol % cholesterol, the selected proportion being adjusted for the most efficacious therapy.

PEGylated peptides having a longer life can also be employed, based on, for example, the conjugate technology described in PCT International Publication No. WO 95/32003.

For parenteral administration, in one embodiment, the IGF agonist compound is formulated generally by mixing each at the desired degree of purity, in a unit dosage injectable form (solution, suspension, or emulsion), with a pharmaceutically, or parenterally, acceptable carrier (i.e. one that is non-toxic to recipients at the dosages and concentrations employed and is compatible with other ingredients of the formulation). For example, the formulation preferably does not include oxidizing agents and other compounds that are known to be deleterious to polypeptides.

Generally, the formulations are prepared by contacting the IGF agonist compound uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, a buffered solution, and dextrose solution. Non-aqueous vehicles such as fixed oils and ethyl oleate are also useful herein.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides (e.g. polyarginine or tripeptides; proteins, such as serum albumin, gelatin, or immunoglobulins); hydrophilic polymers such as polyvinylpyrrolidone; glycine; amino acids such as glutamic acid, aspartic acid, histidine, or arginine; monosaccharides, disaccharides, and other carbohydrates including cellulose or its derivatives, glucose, mannose, trehalose, or dextrins; chelating agents such as EDTA; sugar alcohols such as mannitol or sorbitol; counter-ions such as sodium; non-ionic surfactants such as polysorbates, poloxamers, or polyethylene glycol (PEG); and/or neutral salts, such as, NaCl, KCl, MgCl₂, CaCl₂, etc.

The IGF agonist compound is typically formulated in such vehicles at a pH of from or about 4.5 to about 8. It will be understood that use of certain of the foregoing excipients, carriers, or stabilizers will result in the formation of salts of the compound. The final preparation may be a stable liquid or lyophilized solid.

Typical formulations of the peptide or oral secretagogues as pharmaceutical compositions are discussed below. About 0.5 to about 500 mg of the compound or mixture of compounds, as the free-acid or -base form or as a pharmaceutically acceptable salt, is compounded with a physiologically acceptable vehicle, carrier, excipient, binder, preservative, stabilizer, flavor, etc., as called for by accepted pharmaceutical practice. The amount of active ingredient in these compositions is such that a suitable dosage in the range indicated above is obtained.

Typical adjuvants which may be incorporated into tablets, capsules, and the like are a binder such as acacia, corn starch, or gelatin; an excipient such as microcrystalline cellulose; a disintegrating agent like corn starch or alginic acid; a lubricant such as magnesium stearate; a sweetening agent such as sucrose or lactose; a flavoring agent such as peppermint, wintergreen, or cherry. When the dosage form is a capsule, in addition to the above materials, it may also contain a liquid carrier such as a fatty oil. Other materials of various types may be used as coatings or as modifiers of the physical form of the dosage unit. A syrup or elixir may contain the active compound, a sweetener such as sucrose, preservatives like propyl paraben, a coloring agent, and a flavoring agent such as cherry. Sterile compositions for injection can be formulated according to conventional pharmaceutical practice. For example, dissolution or suspension of the active compound in a vehicle such as water or naturally occumng vegetable oil like sesame, peanut, or cottonseed oil or a synthetic fatty vehicle like ethyl oleate or the like may be desired. Buffers, preservatives, antioxidants, and the like can be incorporated according to accepted pharmaceutical practice.

The IGF agonist compound to be used for therapeutic administration must be sterile. Sterility is readily accomplished by filtration through sterile filtration membranes (e.g., 0.2 μm membranes). Therapeutic compositions generally are placed into a container having a sterile access port, for example, an intravenous solution bag or vial having a stopper pierceable by a hypodermic injection needle.

The IGF agonist compound ordinarily will be stored in unit or multi-dose containers, for example, sealed ampules or vials, as an aqueous solution or as a lyophilized formulation for reconstitution. As an example of a lyophilized formulation, 10-mL vials are filled with 5 ml of sterile-filtered 1% (w/v) aqueous solution of compound, and the resulting mixture is lyophilized. The infusion solution is prepared by reconstituting the lyophilized compound using bacteriostatic Water-for-Injection.

Combination therapy with the IGF agonist compound herein and one or more other appropriate reagents that increase total IGF in the blood or enhance the effect of the IGF agonist is also part of this invention. These reagents generally allow the IGF agonist compound herein to release the generated IGF, and include growth-promoting agents.

Growth-promoting agents for this purpose include, but are not limited to, GH secretagogues that promote the release of endogenous GH in mammals to increase concentrations of the IGF in the blood. Examples include TRH, diethylstilbestrol, theophylline, enkephalins, E series prostaglandins, peptides of the VIP-secretin-glucagon-GRF family, and other GH secretagogues such as GHRP-6, GHRP-1 as described in U.S. Pat. No. 4,411,890, and benzo-fused lactams such as those disclosed in U.S. Pat. No. 5,206,235. (See also, for example, PCT International Publication No. WO 96/15148. Other growth-promoting agents include GHRPs, GHRFs, GH, and their analogs. For example, GHRPs are described in PCT International Publication Nos. WO 95/17422 and WO 95/17423 and in Bowers, (1993) “GH releasing peptides -structure and kinetics”, J. Ped. Endocnnol. 6: 21-31. GHRFs and their analogs are described in, for example, PCT International Publication No. WO 96/37514.

Additionally, GHRH, any of the IGFBPs, long-acting GH, GH plus GHBP, insulin, or a hypoglycemic agent can be employed in conjunction with the IGF agonist compound herein for this purpose. In addition, IGF-I or IGF-II or an IGF with an IGFBP such as IGF-I complexed to IGFBP-3 can also be employed with the IGF agonist compound herein. For example, pharmaceutical compositions containing IGF-I and IGFBP in a carrier as described in PCT International Publication No. WO 94/16723, published Aug. 4, 1994, can be used in conjunction with the compound. The entities can be administered sequentially or simultaneously with the IGF agonist compound. In addition, other means of manipulating IGF status, such as regimens of diet or exercise, are also considered to be combination treatments as part of this invention.

In addition, the invention contemplates using gene therapy for treating a mammal, using nucleic acid encoding the IGF agonist compound, if it is a peptide. Generally, gene therapy is used to increase (or overexpress) IGF levels in the mammal. Nucleic acids which encode the IGF agonist peptide can be used for this purpose. Once the amino acid sequence is known, one can generate several nucleic acid molecules using the degeneracy of the genetic code, and select which to use for gene therapy.

There are two major approaches to getting the nucleic acid (optionally contained in a vector) into the patient's cells for purposes of gene therapy: in vivo and ex vivo. For in vivo delivery, the nucleic acid is injected directly into the patient, usually at the site where the IGF agonist compound is required. For ex vivo treatment, the patient's cells are removed, the nucleic acid is introduced into these isolated cells, and the modified cells are administered to the patient either directly or, for example, encapsulated within porous membranes which are implanted into the patient. (See also, for example, U.S. Pat. Nos. 4,892,538 and 5,283,187.)

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. A commonly used vector for ex vivo delivery of the gene is a retrovirus.

The currently preferred in vivo nucleic acid transfer techniques include transfection with viral vectors (such as adenovirus, Herpes simplex I virus, or adeno-associated virus) and lipid-based systems (useful lipids for lipid-mediated transfer of the gene are DOTMA, DOPE and DC-Chol, for example). In some situations it is desirable to provide the nucleic acid source with an agent that targets the target cells, such as an antibody specific for a cell-surface membrane protein or the target cell, a ligand for a receptor on the target cell, etc. Where liposomes are employed, proteins which bind to a cell-surface membrane protein associated with endocytosis may be used for targeting and/or to facilitate uptake, e.g., capsid proteins or fragments thereof tropic for a particular cell type, antibodies for proteins which undergo internalization in cycling, and proteins that target intracellular localization and enhance intracellular half-life. The technique of receptor-mediated endocytosis is described, for example, by Wu et al., (1987) “Receptor-mediated in vitro gene transformation by a soluble DNA carrier system”, J. Biol. Cliem. 262: 4429-4432, and Wagner et al., (1990) “Transferrin-polycation conjugates as carriers for DNA uptake into cells”, Proc. Nat'l Acad. Sci. USA . 87: 3410-3414. For review of the currently known gene marking and gene therapy protocols, see Anderson, WF (1992) Human gene therapy. Science 256: 808-813; and also PCT International Publication No. WO 93/25673 and the references cited therein.)

Kits are also contemplated for this invention. A typical kit would comprise a container, preferably a vial, for the IGF agonist compound formulation comprising IGF agonist compound in a pharmaceutically acceptable buffer and instructions, such as a product insert or label, directing the user to utilize the pharmaceutical formulation. The kit optionally includes a container, preferably a vial, for a GH, a GHRP, a GHRH, a GH secretagogue, an IGF, an IGF complexed to an IGFBP, an IGFBP, a GH complexed with a GHBP, insulin, or a hypoglycemic agent.

Also provided is a method for predicting the relative affinity for binding to a ligand of a peptide that competes with a polypeptide for binding to the ligand, which peptide is derived from a phage-displayed library, which method comprises incubating a phagemid clone corresponding to the peptide with the polypeptide in the presence of the ligand, serially diluting the phage, and measuring the degree to which binding of the phagemid clone to the ligand is inhibited by the peptide, wherein a phagemid clone that is inhibited only at low phage concentrations has a higher affinity for the ligand than a phagemid clone that is inhibited at both high and low phage concentrations. An example of such a method may be found, for example, in Example 7 of U.S. Pat. No. 6,121,416. Preferably, the ligand is an IGFBP such as IGFBP-1 or IGFBP-3 and the polypeptide is an IGF.

In another embodiment herein, a method is provided for directing endogenous IGF either away from, or towards, a particular site in a mammal comprising administering to the mammal an effective amount of the compound herein that is specific for an IGFBP that is either prevalent at, or absent from, the site. “Sites” for this purpose include specific tissues or organs such as the heart, or such as the brain via brain-specific IGFBPs. Prevalence at the site indicates that the IGFBP in question is located at the site and constitutes a substantial or biologically important portion of the IGFBP at the site. This indication follows from the specificity for IGFBP-1 versus IGFBP-3 of the compounds demonstrated herein.

Doses of consensus interferon (Infergen™, interferon alfacon-1) in the range of about 5 μg to about 15 μg administered subcutaneously 3 times weekly (see U.S. Pat. No. 5,980,884) are suitable.

### EXAMPLE 1

### Materials and Methods

The following experimental protocol followed guidelines approved by the University of Auckland Animal Ethics Committee.

### Animals and Surgery

Twenty one Romney/Suffolk fetal sheep were instrumented at 117-124 days of gestation (term=147 days) under general anaesthesia (2% halothane in 02) using sterile techniques (Guan et al., 2000; Gunn et al., 1997). Ewes were given 5 ml of Streptopen™ intramuscularly for prophylaxis. Polivinyl catheters were placed in both brachial arteries. The vertebral-occipital anastomoses were ligated bilaterally to restrict vertebral blood supply to the carotid arteries. A double-ballooned inflatable occluder cuff was placed around each carotid artery. Two pairs of electroencephalographic (EEG) electrodes (AS633-5SSF, Cooner Wire Co., Chatsworth, Calif., USA) were placed on the dura over the parasagittal parietal cortex (5 mm and 15 mm anterior to bregma and 10 mm lateral), with a reference electrode sewn to the occiput (Gunn et al., 1997). To record cortical impedance, a third pair of electrodes (Cooner Wire AS 633-3SSF) was placed over the dura 5 mm lateral to the EEG electrodes. A 17 mm long cannula was inserted into the left lateral cerebral ventricle at 4 mm anterior and 6 mm lateral to bregma (Guan et al., 2000). The fetus was then returned to the uterus and gentamicin (80 mg) was administered into the amniotic sac prior to closure of the uterus. All catheters and electrodes were exteriorised through the maternal flank. A maternal femoral vein was catheterised.

Post-surgery sheep were housed together in separate metabolic cages with access to water and food ad libitum. They were kept in a temperature controlled room (16° C., 50% humidity), in a 12 hour day/night cycle. A period of 3 days post-operative recovery was allowed during which time antibiotics were administered daily to the ewe (600 mg Crystapen™ intravenously for 4 days and 80 mg gentamicin, intravenously daily for the first 3 days). Fetal arterial blood was taken daily for blood gas analysis. Vascular catheters were maintained patent by continuous infuision of heparinized saline (40 U/ml at 0.2ml/h). The lateral ventricle cannula was maintained patent by daily flushing with 200 μl of artificial CSF (Guan et al., 2000).

### Experimental Procedures

Fetuses were randomly assigned to either sham ischemia with no infusion (sham control group, n=4), or ischemia groups who received intracerebroventricular (i.c.v.) infusions of 1 ml of either vehicle (artificial CSF, n=8) or 3 μg rhlGF-I (n=9), kindly provided by Dr D. Hung (Chiron, Emeryville, Calif., USA). Reversible cerebral ischemia was induced by inflation of both carotid cuffs with sterile saline for 30 minutes. Successful occlusion was confirmed by the onset of an isoelectric EEG signal within 30 sec of inflation (Gunn et al., 1997). Before administration, the pH of the infusate was buffered to 7.33-7.39 with 1M NaHCO₃, as previously described (Johnstone et al., 1996). The dead space in the lateral ventricle catheter (0.7 ml) was primed with either rhIGF-I or vehicle by infusion over 45 minutes, 90 minutes after the reperfusion, IGF-I or vehicle were infused over 1 hour. At the end of the experiment, 96 hour after ischemia, the ewe and fetus were killed by an intravenous overdose of pentobarbital. The fetus was rapidly removed through an abdominal incision, and the brain perfusion fixed in situ with normal saline, followed by 10% phosphate buffered formalin. Each brain was removed from the skull and fixed in the same fixative for a further 7 days before processing and embedding using a standard paraffin tissue preparation.

### Immunohistochemistry

The following primary antibodies were used: rabbit antisera raised against myelin basic protein (MBP, Roche, Mannheim, Germany) to label myelin; isolectin B-4 (Sigma, St Louis, Miss., USA) to label reactive microglia; glial fibrillary acidic protein (GFAP, Sigma) to label reactive astrocytes. The antibodies were diluted in 1% goat serum in PBS and 0.4% merthiolate.

Immunohistochemical staining was performed on coronal sections (6 μm), at the level of the parietal cortex, cut and mounted on chrome alum coated slides. The sections were deparaffinized in xylene, dehydrated in a series of ethanol and incubated in PBS (0.1M). The sections were pretreated with 1% H₂0₂ in 50% methanol for 20 minutes, washed in PBS (3×10 minutes), and then incubated for 2 days at 4° C. in the following primary antibodies at the dilutions indicated: MBP (1:200), isolectin B-4 (1:100), and GFAP (1:200). The primary antibodies were washed off with PBS (3×10 minutes) and then incubated with goat anti-rabbit biotinylated secondary antibody (1:200, Sigma) overnight at room temperature. The sections were washed, incubated in ExtrAvidin™ (Sigma, 1:200) for 3 hours, washed again in PBS-triton and then reacted in 0.05% 3,3-diaminobenzidine tetrahydrochloride (DAB) and 0.01% H₂O₂ to produce a brown reaction product, dehydrated in a series of alcohol to xylene and coverslipped with mounting medium. Control sections were processed in the same way except that the primary antibody was omitted from the incubating solution. Adjacent sections were also stained with thionin in order to examine apoptotic morphology.

### Cloning of Sheep Proteolipidprotein (PLP) Gene

PLP expression is a biological marker for myelination at the transcript level. Five μg of total RNA extracted from near term fetal sheep cortex was used to synthesize a single strand cDNA using SuperscriptII RNaseH Reverse transcriptase (Gibco BRL, Gaithesburg, Md., USA). A 417 base pair fragment corresponding to nucleotide 79 to 495 of bovine partial PLP mRNA sequence (Genebank) was generated by polymerase chain reaction (PCR) with the following primers: upper 5′-ACCTATGCCCTGACCGTTG-3′, lower 5′-TGTGTGGTTAGAGCCTCGC-3′. The PCR conditions were: 3 minutes at 94° C.; 35 cycles of 30 seconds at 94° C., 30 seconds at 58° C., 7 minutes at 72° C. The PCR product, which formed a single band at the expected size when resolved in agarose gel electrophoresis, was subcloned into plasmid pCR2. 1 (Invitrogen, Carlsbad, Calif., USA) by TA cloning and sequenced from both M13 reverse and forward directions. DNA sequencing identified the cloned fragment as sheep PLP. Compared with mRNA sequences of other species in the Genebank, the cloned sheep PLP sequence shared 100%, 95.7% and 94.2% similarity with bovine, human and rat PLP respectively at the nucleotide level. The sheep PLP cDNA fragment was released from the flanking EcoRI sites and rebsubcloned into the EcoRI site of pBluescriptIIKS (Stratagene, La Jolla, Calif., USA), which was used as a template to make RNA probes.

### In situ Hybridization

Antisense or sense digoxigenin (DIG)-labeled RNA probes were synthesized by in vitro transcription from HindIII or BamHI linearized template using DIG RNA Labeling Mix (Boehringer Mannheim) and T7 or T3 RNA polymerase (Gibco BRL) respectively according to Boehringer Mannheim's instruction.

In situ hybridization was carried out as described previously (Lai et al., 1996). Briefly, before hybridization, paraffin embedded sections (6 μm) were dewaxed, rehydrated and subjected to post-fixation, proteinase K treatment and acetylation treatment sequentially. Dehydrated and air-dried sections were hybridized with probes in a humidified box at 58° C. overnight. After hybridization, sections were treated with RNAse A and washed to 0.1×SSC/DTT at 65° C. for 30 minutes. The sections were then washed for 20 minutes with MABT buffer containing maleic acid (100 mM), NaCl (150 mM) and Tween 20 (0.1%, pH 7.5), 3 times. The sections were incubated with block solution containing normal sheep serum (10%) and 2% blocking agent (Boehringer Mannheim), made with MABT buffer, at room temperature for one hour and incubated with sheep anti-DIG-alkaline phosphatase Fab fragment (Boeringer Mannheim), diluted 1:300 in block solution, at 4° C. overnight. Sections were washed with MABT buffer containing 2 mM levamisole (Sigma) for 3 times and freshly prepared staining buffer containing 100 mM NaCl, 100 mM Tris (pH9.5), 50 mM MgCl₂, 0.1% Tween 20 and 2 mM levamisole for 3 times. Signals were visualized with 225 μg/ml 4-nitro blue tetrazolium chloride (NBT, Promega) and 175 μg/ml 5-bromo-4-choro-3-indoyl-phosphate (BCIP, Promega) diluted in staining buffer at RT in a light-tight box for 3 hours. The slides were coverslipped for microscopy or used for immunohistochemical staining.

### Analysis

The numbers of isolectin B-4, GFAP and PLP positive cells were counted in three areas in the intragyral white matter of the parasagittal cortex and one area in the corona radiata of both sides by light microscopy (×20). The counts in the three intragyral areas were averaged. The number of positive cells was then converted to number of cells/mm2 for comparison between the vehicle and IGF-1 treated groups, using two way ANOVA, with sampling area (intragyral white matter and corona radiata) treated as a repeated measure (SPSS v10, SPSS Inc, Chicago, Ill.). Where a significant effect of either group or an interaction between group and area was found, further post-hoc comparisons were performed using the least difference test. The density of MBP from the same areas and their background was measured by image analysis (Sigmascan, SPSS Inc, Chicago, Ill.). The difference between the MBP density and the background reading from adjacent grey matter was calculated and used for data analysis. The effect of IGF-1 treatment on the density of MBP was then assessed using two way ANOVA, after transformation to normalise the data. The relationship between MBP density and numbers of PLP positive cells was examined by backward stepwise regression (SPSS). The co-localisation of PLP positive cells with GFAP and isolectinB-4, as well as cells with apoptotic morphology were examined and photographed by light microscopy (Nikon E800, Nikon, Tokyo, Japan).

### Results

Sheep brains were examined as described to assess the effects of experimental treatments on remyelination. The corona radiata and intragyral regions of sheep brains were inspected to assess the number of cells expressing proteolipid protein mRNA (PLP mRNA, to label bioactive oligodendrocytes and myelination at the transcript level), glial fibrillary acid protein (GFAP, to label reactive astrocytes) and isolectin B-4 (to label reactive microglia) immunopositivity and the average density of myelin basic protein (MBP, to label myelin) in the intragyral white matter and the corona radiata. One area in the corona radiata and three regions from the intragyral white matter of both hemispheres were used for assessment (areas were 1 mm²).

GFAP immunohistochemical counter-staining with PLP mRNA DIG demonstrated that some PLP mRNA positive cells were co-localised with GFAP immunopositive staining. Such co-localization of DIG labeling for PLP mRNA with GFAP immunopositive staining was common. PLP/GFAP double positive astrocytes were found throughout both the corona radiata and the intragyral tracts. There are two distinct morphological relationships between PLP and GFAP. In the first type of co-localisation PLP and GFAP staining are located in two separate cells. It thus appears that PLP positive cells were co-localised with astrocytes. In the second type, the PLP positivity was localised within the same cells that also expressed GFAP positive staining in the processes, that is “co-expression”. Within the most severely damage tissues, the PLP/GFAP positive cells were more isolated and had fewer and shorter GFAP positive processes; these cells clearly showed type one co-localisation.

TABLE 1
Effects of IGF-I on PLP mRNA expression. The number of cells
expressing PLP mRNA was counted in the corona radiata and
intragyral cerebral white matter tracts. Data are mean ± SD
cells/mm².

| | Sham Controls | Ischemia + Vehicle | Ischemia + IGF-I |
|---|---|---|---|
| | n = 4 | n = 8 | n = 9 |
| Corona radiata | 131.87 ± 45.1 | 66.3 ± 64.0 | 109.8 ± 37.2 |
| Inragyral | 85.66 ± 22.9 | 23.7 ± 23.5# | 54.3 ± 27.4* |

# p < 0.01 sham control vs. ischemia + vehicle
* p < 0.05 ischemia + vehicle vs. ischemia + IGF-I treated groups.

TABLE 2
Effect of IGF-I on MBP density. The average density of MBP
immunostaining was measured in the corona radiata and the
intragyral cerebral white matter tracts using image analysis.

| | Sham Controls | Ischemia + Vehicle | Ischemia + IGF-I |
|---|---|---|---|
| | N = 4 | N = 8 | N = 8 |
| Corona radiata | 34.7 ± 10.8 | 23.8 ± 24.9 | 32.6 ± 15.9 |
| Inragyral | 29.3 ± 9.4 | 6.3 ± 16.7# | 18.8 ± 15.6* |

# p < 0.01 sham control vs. ischemia + vehicle
* p < 0.05 ischemia + vehicle vs. ischemia + IGF-I. Data are mean ± SD.

TABLE 3
Effect of IGF-I on reactive glia. The number of GFAP or isolectin
B-4 immunopositive cells were counted in the corona radiata and
intragyral cerebral white matter tracts. Data are mean ± SD
cells/mm².

| | Isehemia + Vehicle | | Ischemia + IGF-I | |
|---|---|---|---|---|
| | GFAP | Isolectin | GFAP | Isolectin |
| corona | 59.2 ± 17.1 | 46.6 ± 16.5 | 78.2 ± 20.4 | 82.7 ± 24.6* |
| radiata | | | | |
| Intragyral | 40.4 ± 7.5 | 40.7 ± 13.7 | 65.6 ± 12.5** | 72.5 ± 24.0* |

* p < 0.05,
** p < 0.001 ischemia + vehicle vs. ischemia + IGF-I.

### Conclusion

These results demonstrate that IGF-I induces astrocytes to promote remyelination after brain injury and thus demonstrates that astrocytes are able to contribute to remyelination.

### EXAMPLE 2

IGF-I and/or a biologically active analog of IGF-I analog and/or biologically active IGF-I mimetic in the amount from about 0.1 to 1000 μg of IGF-I per 100 g of body weight are administered via lateral cerebro-ventricular injection, focal injection, or a surgically inserted shunt into the lateral cerebro-ventricle of the brain of the patient and/or IGF-I (from about 0.1 to 1000 μg of IGF-I per 100 g of body weight administered via lateral cerebro-ventricular injection, focal injection, or a surgically inserted shunt into the lateral cerebro-ventricle of the brain of the patient and catheter to promote) in combination with interferon β1b (Betaseron™) (from about 0.006 mg to 2.0 mg) are administered iv to promote remyelination in periods of symptom relapse in relapsing-remitting multiple sclerosis.

### EXAMPLE 2A

IGF-I in the amount of 10 mg of IGF-I per 100 g of body weight is administered via lateral cerebro-ventricular injection into the lateral cerebro-ventricle of the brain of a patient in combination with consensus interferon (Infergen™, Interferon alfacon-1) (12 μg) administered subcutaneously to promote remyelination in periods of symptom relapse in relapsing-remitting multiple sclerosis. Remyelination is promoted by the treatment.

### EXAMPLE 2B

A biologically active analog of IGF-I analog in the amount of 100 mg of IGF-I analog per 100 g of body weight is administered via focal injection into the lateral cerebro-ventricle of the brain of the patient in combination with consensus interferon (Infergen™, Interferon alfacon-1) (15 μg) administered subcutaneously to promote remyelination in periods of symptom relapse in relapsing-remitting multiple sclerosis. Remyelination is promoted by the treatment.

### EXAMPLE 2C

IGF-I and a biologically active IGF-I mimetic in the amount of 1 mg of IGF-I and 100 m g of biologically active IGF-I mimetic per 100 g of body weight is administered via a surgically inserted shunt into the lateral cerebro-ventricle of the brain of the patient in combination with consensus interferon (Infergen™, Interferon alfacon-1) (9 μg) administered subcutaneously to promote remyelination in periods of symptom relapse in relapsing-remitting multiple sclerosis. Remyelination is promoted by the treatment.

### EXAMPLE 2D

IGF-I and a biologically active IGF-I mimetic in the amount of 1 mg of IGF-I and 100 mg of biologically active IGF-I mimetic per 100 g of body weight are administered via a surgically inserted shunt into the lateral cerebro-ventricle of the brain of the patient in combination with consensus interferon (Infergen™, Interferon alfacon-1) (9 μg) which is administered subcutaneously 3 times a week (at least 48 hours between doses) to promote remyelination in periods of symptom relapse in relapsing-remitting multiple sclerosis. Remyelination is promoted by the treatment.

### EXAMPLE 3

Twenty one sheep are treated according to the method of Example 1. Fetuses are randomly assigned to either sham ischemia with no infusion (sham control group, n=4), or ischemia groups who receive intracerebroventricular (i.c.v.) infusions of 1 ml of either vehicle (artificial CSF, n=8) or 3 μg rhIGF-I plus Interferon beta Ib (betaseron) 0.1 mg (n=9). rhlGF-I is available from Chiron, Emeryville, Calif., USA, and Betaseron is available from Berlex Laboratories, Richmond, Calif., USA. Reversible cerebral ischemia is induced by inflation of both carotid cuffs with sterile saline for 30 minutes. Successful occlusion is confirmed by the onset of an isoelectric EEG signal within 30 sec of inflation (Gunn et al., 1997). Before administration, the pH of the infusate is buffered to 7.33-7.39 with IM NaHCO₃, as previously described (Johnstone et al., 1996). The dead space in the lateral ventricle catheter (0.7 ml) is primed with either rhlGF-I or vehicle by infusion over 45 minutes, 90 minutes after the reperfusion, IGF-I or vehicle are infused over 1 hour. At the end of the experiment, 96 hour after ischemia, the ewe and fetus are killed by an intravenous overdose of pentobarbital. The fetus is rapidly removed through an abdominal incision, and the brain perfusion fixed in situ with normal saline, followed by 10% phosphate buffered formalin. Each brain is removed from the skull and fixed in the same fixative for a further 7 days before processing and embedding using a standard paraffin tissue preparation. Induction of remyelination is demonstrated.

It will be appreciated by those persons skilled in the art that the above description is provided by way of example only and that numerous changes and variations can be made while still being with the scope of the invention as defined by the appended claims.

We claim:

1. A method of restoring myelination of axons by stimulating glial cells other than oligodendrocytes to promote remyelination in an animal in need of restored myelination due to neural injury or disease, comprising administering to the animal a therapeutic amount effective to restore myelination of axons in the animal of an Insulin-like Growth Factor-I (IGF-I) compound, where the IGF-I compound is selected from the group consisting of IGF-I, a biologically active IGF-I analog, a biologically active IGF-I mimetic, a compound that increases the concentration of IGF-I, and a compound that increases the concentration of IGF-I analogs.

2. The method of claim 1, where the IGF-I compound is effective to stimulate astrocytes to produce myelin.

3. The method of claim 1, where the IGF-I compound is effective to stimulate satellite cells to promote remyelination.

4. A method of restoring myelination of axons by stimulating glial cells other than oligodendrocytes to promote remyelination in an animal in need of restored myelination due to neural injury or disease, comprising administering a therapeutic amount effective to restore myelination of axons in the animal of an IGF-I compound in combination with an interferon, where the IGF-I compound is selected from the group consisting of IGF-I, a biologically active IGF-I analog, a biologically active IGF-I mimetic, and a compound that increases the concentration of IGF-I or IGF-I analogs.

5. The method of claim 4, where the combination of IGF-I compound and interferon is effective to stimulate astrocytes to produce myelin.

6. The method of claim 4, where the combination of IGF-I compound and interferon is effective to stimulate satellite cells to promote remyelination.

7. The method of claims 1 or 4, where the neural injury or disease is a disease or disorder selected from the group consisting of trauma, toxin exposure, asphyxia or hypoxia-ischemia, perinatal hypoxic-ischemic injury, injury to or disease of the white matter of the central nervous system, acute brain injury, chronic neurodegenerative disease, and demyelinating diseases and disorders.

8. The method of claim 7, where the neural injury or disease is multiple sclerosis.

9. The method of claim 7, where the neural injury or disease is selected from the group consisting of acute disseminated encephalomyelitis, optic neuritis, transverse myelitis, Devic's disease, the leukodystrophies, progressive multifocal leukoencephalopathy, and central pontine myelinolysis.

10. The method of claims 1 or 4, where the IGF-I compound is IGF-I.

11. The method of claims 1 or 4, where the IGF-I compound is an IGF-I analog.

12. The method of claim 11, where the IGF-I compound is selected from the group consisting of insulin-like growth factor 2 (IGF-2) and truncated IGF-I (des 1-3 IGF-I).

13. The method of claims 1 or 4, where the IGF-I compound is an IGF-I mimetic.

14. The method of claim 13, where the IGF-I compound is selected from the group consisting of IGFBP-1 binding peptide p1-01 and insulin-like growth factor agonist molecules.

15. The method of claims 1 or 4, where the IGF-I compound is a compound that increases the concentration of IGF-I or IGF-I analogs in the animal.

16. The method of claim 4, where the interferon (IFN) is selected from the group consisting of IFN-α, IFN-β, IFN-ω, consensus-IFN, and combinations thereof.

17. The method of claim 16, where the interferon is an interferon-β.

18. The method of claim 17, where the interferon-β is interferon-β1b.

19. The method of claim 16, where the interferon is consensus interferon.

20. The method of claim 19, where the consensus interferon is selected from the group consisting of IFN-con₁, IFN-con₂, and IFN-con₃.

21. The method of claims 1 or 4, where the neural injury or disease is central nervous system hypoxic injury.

22. The method of claims 1 or 4, where the neural injury or disease is central nervous system ischemic injury.

23. The method of claims 1 or 4, where the neural injury or disease is peripheral nervous system hypoxic injury.

24. The method of claims 1 or 4, where the neural injury or disease is peripheral nervous system ischemic injury.

25. The method of claims 1 or 4, where the neural injury or disease is central nervous system injury as a consequence of multiple sclerosis.

26. The method of claims 1 or 4, where the neural injury or disease is central nervous system injury as a consequence of a demyelinating disorder.

27. The method of claims 1 or 4, where the neural injury or disease is peripheral nervous system injury as a consequence of multiple sclerosis.

28. The method of claims 1 or 4, where the neural injury or disease is peripheral nervous system injury as a consequence of a demyelinating disorder.

29. The method of claims 1 or 4, where the step of administering a therapeutic amount of an IGF-I compound comprises introducing a nucleic acid encoding an IGF-I compound into the animal.

30. The method of claims 1 or 4, further comprising administering a therapeutically effective amount of a growth-promoting agent, where the growth-promoting agent is selected from the group consisting of growth hormone, growth hormone analogs, growth hormone mimetics, agents that increase the concentration of growth hormone in the blood of an animal, and growth hormone secretagogues.

31. The method of claims 1 or 4, where the neural injury or disease is central nervous system injury and where the IGF-I compound is administered in the period from the time of central nervous system injury to about 100 hours after the injury.

32. The method of claim 31, where the neural injury or disease is central nervous system injury and where the IGF-I compound is administered at least once in the period from the time of the central nervous system injury to about 8 hours subsequently.

33. The method of claims 1 or 4, where the neural injury or disease is peripheral nervous system injury and where the IGF-I compound is administered in the period from the time of central nervous system injury to about 100 hours after the injury.

34. The method of claim 33, where the neural injury or disease is peripheral nervous system injury and where the IGF-I compound is administered at least once in the period from the time of the central nervous system injury to about 8 hours subsequently.

35. The method of claim 4, where the neural injury or disease is central nervous system injury and where the IGF-I compound in combination with interferon β1b is administered in the period from the time of the central nervous system injury to about 100 hours after the injury.

36. The method of claim 4, where the neural injury or disease is central nervous system injury and where the IGF-I compound in combination with consensus interferon is administered in the period from the time of the central nervous system injury to about 100 hours after the injury.

37. The method of claim 4, where the neural injury or disease is peripheral nervous system injury and where the IGF-I compound in combination with interferon β1b is administered in the period from the time of the central nervous system injury to about 100 hours after the injury.

38. The method of claim 4, where the neural injury or disease is peripheral nervous system injury and where the IGF-I compound in combination with consensus interferon is administered in the period from the time of the central nervous system injury to about 100 hours after the injury.

39. The method of claims 1 or 4, where the IGF-I compound is administered to the animal in an amount from about 0.1 μg to about 1000 μg of IGF-I compound per 100 g of body weight of the animal.

40. The method of claims 1 or 4, where the IGF-I compound is administered to the animal through a surgically inserted shunt into a ventricle of the animal.

41. The method of claims 1 or 4, where the IGF-I compound is administered peripherally into the animal.

42. A kit comprising an IGF-I compound formulated in a pharmaceutically acceptable buffer, a container for holding the IGF-I compound formulated in the pharmaceutically acceptable buffer, and instructions.

43. The kit of claim 42, further comprising a compound selected from the group consisting of growth hormone, a growth hormone releasing protein, a growth hormone releasing hormone, a growth hormone secretagogue, and a growth hormone complexed with a growth hormone binding protein.

44. The kit of claim 42, further comprising a compound selected from the group consisting of an IGF binding protein, an IGF-I compound complexed to an IGF binding protein, insulin, and a hypoglycemic agent.
